(19) United States
(12) Patent Application Publication
(10) Pub. No.: US 2026/0023004 A1
(43) Pub. Date: Jan. 22, 2026

(54) SIGNAL ENHANCEMENT OF RESONANT SENSOR FOR CELL MEASUREMENTS

(71) Applicant: lowa State University Research Foundation, Inc., Ames, IA (US)

(72) Inventors: Nigel Forest Reuel, Ames, IA (US); Yee Jher Chan, Ames, IA (US)

(21) Appl. No.: 19/112,123

(22) PCT Filed: Jun. 7, 2023
(86) PCT No.: PCT/US2023/024675

§ 371 Date: Mar. 14, 2025

Related U.S. Application Data
(60) Provisional application No. 63/375,997, filed on Sep. 16, 2022.

(51) Int. Cl. G01N 15/06; G01N 15/01; G01N 33/483

(52) U.S. Cl. CPC G01N 15/0656; G01N 15/01; G01N 33/4833

(57) ABSTRACT

A variety of applications can include cell-responsive structures to provide enhanced sensitivity of a resonant sensor in cell detection. A cell-responsive layer can be structured over a resonant sensor. The arrangement of the resonant sensor and the cell-responsive layer can be implemented as a resonant sensor to measure changes in cells. With the cell-responsive layer responsive to cells proximate to the cell-responsive layer, changes in resonant frequency of the arrangement of the resonant sensor and the cell-responsive layer over time at which the cells are proximate to the cell-responsive layer can be monitored. Interrogating the resonant sensor can be conducted wirelessly along with wirelessly transmitting data collected from the interrogation. Additional apparatus, systems, and methods are disclosed.

### CLAIM OF PRIORITY

This application claims the priority benefit of U.S. Provisional Application Ser. No. 63/375,997, filed 16 Sep. 2022, which application is incorporated herein by reference in its entirety.

### STATEMENT OF GOVERNMENT SUPPORT

This invention was made with government support under Contract No. CBET2042503 awarded by the National Science Foundation. The government has certain rights in the invention.

### FIELD OF THE INVENTION

The invention relates generally to monitoring technologies, in particular, devices and monitoring associated with measuring of cells or similar structures.

### BACKGROUND

Non-invasive measurements of a cell culture enable more controls on a system, associated with the cell culture, that provide benefits in both research and industry. Resonant sensors are wireless, passive, and cost effective and are potential candidates for these measurements. However, the sensing region of this type of sensor is typically proportional to the resonant sensor size such that a smaller sensor is able to sense a smaller target and vice versa. Therefore, typical resonant sensors that are in the centimeters scale may not be able to sense or exhibit sensitivity towards micrometers size targets, such as cells. In the case of cells, their contribution to permittivity change, with respect to the resonant sensors, is small compared to the larger interrogation zone of the resonant sensor.

### BRIEF DESCRIPTION OF THE FIGURES

The drawings, which are not necessarily drawn to scale, illustrate generally, by way of example, but not by way of limitation, various embodiments discussed in the present document.

FIGS. 1-2 illustrate a schematic of a sensor prototype initially prototyped in a petri dish for a sensor system, in accordance with various embodiments.

FIG. 3 is an representation of an image of the petri dish having the sensor prototype of FIGS. 1-2, in accordance with various embodiments.

FIG. 4 is a representation of the prototype sensor system of FIG. 3 as well as an experiment setup that integrates a readout coil and a microscope, in accordance with various embodiments.

FIG. 5 illustrates changes in resonant frequency over time when sensor systems, having a resonant sensor and a cell-responsive layer to the resonant sensor, were cultured with varying cell seeding concentrations in a container containing the resonant sensor and the cell-responsive layer to the resonant sensor, in accordance with various embodiments.

FIGS. 6-10 show changes in resonant frequency correlated with cell images obtained from a microscope, in accordance with various embodiments.

FIGS. 11-15 show changes in resonant frequency correlated with cell images obtained from a microscope, in accordance with various embodiments.

FIG. 16 shows changes in resonant frequency correlated with cell images obtained from a microscope, in accordance with various embodiments.

FIGS. 17 and 18 show results of further investigation performed on cell types and cell-responsive layer thickness, in accordance with various embodiments.

FIG. 19 is a top view of a sensor prototype having a petri dish as a container for a sensor system, in accordance with various embodiments.

FIG. 20 is a side view of the sensor prototype of FIG. 19 at an initial time when a layer of cells is placed on a cell-responsive layer and petri dish, in accordance with various embodiments.

FIG. 21 is a side view of the sensor prototype of FIG. 20 after a period of cell growth, in accordance with various embodiments.

FIG. 22 is a top-down image from a digital microscope of a tape stretched tightly across a gap corresponding to a laser cut gap of FIG. 20, in accordance with various embodiments.

FIG. 23 shows height information of the tape over the laser cut gap of FIG. 22, in accordance with various embodiments.

FIG. 24 is a top-down image from the digital microscope of the tape of FIG. 22 upon cell exposure, which shows the tape sagging, in accordance with various embodiments.

FIG. 25 shows height information of the tape of FIG. 24 that reflects the sagging of the tape of FIG. 24, in accordance with various embodiments.

FIG. 26 shows an arrangement of a resonant sensor and a cell-responsive layer to provide enhanced sensitivity of a resonant sensor in cell detection, in accordance with various embodiments.

FIG. 27 shows another arrangement of a resonant sensor and a cell-responsive layer to provide enhanced sensitivity of a resonant sensor in cell detection, in accordance with various embodiments.

FIG. 28 is a block diagram of an embodiment of an example system architecture to provide enhanced sensitivity of a resonant sensor structure in cell detection, in accordance with various embodiments.

FIG. 29 is a flow diagram of features of an example method of measuring cells, in accordance with various embodiments.

FIG. 30 is a flow diagram of features of an example method of forming a sensor arrangement for wireless measurement of cells, in accordance with various embodiments.

### DETAILED DESCRIPTION

The following detailed description refers to the accompanying drawings that show, by way of illustration, various embodiments of the invention. These embodiments are described in sufficient detail to enable those of ordinary skill in the art to practice these and other embodiments. Other embodiments may be utilized, and structural, logical, mechanical, and electrical changes may be made to these embodiments. The various embodiments are not necessarily mutually exclusive, as some embodiments can be combined with one or more other embodiments to form new embodiments. The following detailed description is, therefore, not to be taken in a limiting sense.

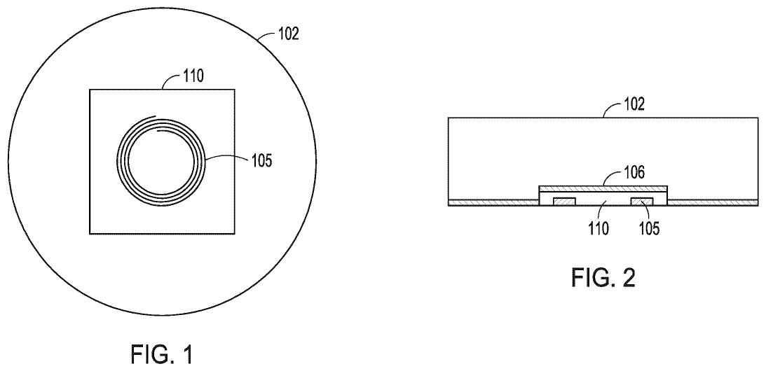

In various embodiments, a cell-responsive layer implemented with a resonant sensor can magnify the signal sensitivity of the resonance response of the cell-responsive layer combined with the resonant sensor as a function of secreted molecules. Herein, a cell is a biological cell. FIGS. 1-2 illustrate a schematic of a sensor prototype initially prototyped in a petri dish 102 for a sensor system. FIG. 1 is a top view of the sensor prototype in petri dish 102 and FIG. 2 is a side view of the sensor prototype in petri dish 102. A resonant sensor in this initial prototype includes a wound copper coil 105 in combination with a cell-responsive layer 110. A cell-responsive layer is a material that can interact or allow material of cells to be absorbed. A cell-responsive layer can be a material that responds to cell growth and can undergo change in its properties. Such changes can include, for example, changes of elasticity that wraps around at least portion of wound copper coil 105 and, therefore, changes the signal provided in response to an interrogation by an external source.

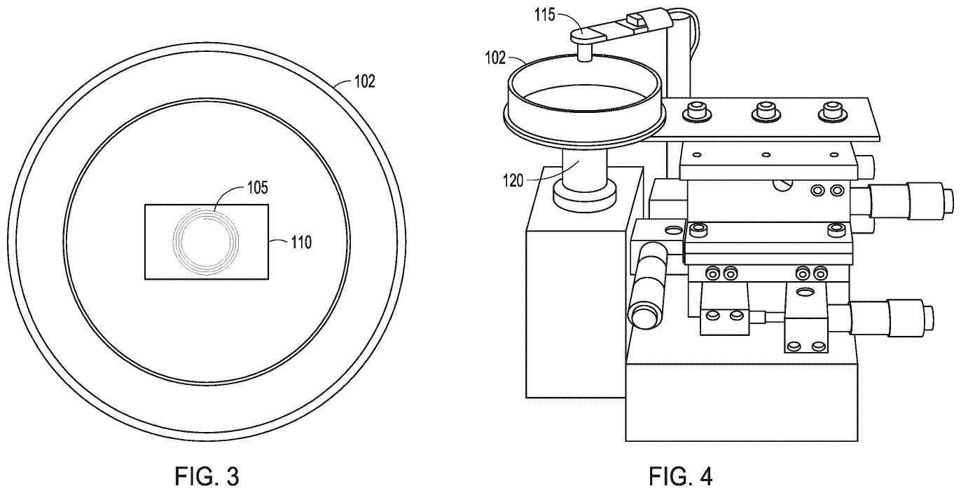

In this prototype, an acrylic adhesive transfer tape was used as cell-responsive layer 110, providing a soft substrate for cells 106 and providing a mechanism to hold copper coil 105 in petri dish 102. The resonant sensor of wound copper coil 105 and cell-responsive layer 110 can be interrogated with cells introduced into petri dish 102. Petri dish 102 may contribute to the resonant sensor provided by combination of wound copper coil 105 and cell-responsive layer 110, where such contribution may be part of a baseline measurement. Depending on the nature of the cells being measured, a sensor system can be structured without a container such as petri dish 102. For example, the cells being measured may be introduced to adhere to and remain on cell-responsive layer 110 without flowing off cell-responsive layer 110. After sterilization by ultraviolet (UV) light, petri dish 102 was ready for cell culture as shown in FIG. 3.

FIG. 3 is a representation of an image of petri dish 102 having the sensor prototype of FIGS. 1-2. For sensor interrogation, an external coil connected to a vector network analyzer was used for obtaining the resonant frequency of the resonant sensor having wound copper coil 105. To correlate the resonant frequency to cell growth, a microscope 120 was integrated into the sensor system as shown in FIG. 4. FIG. 4 is a representation of the prototype sensor system of FIG. 3 as well as an experiment setup that integrates a readout coil 115 and microscope 120.

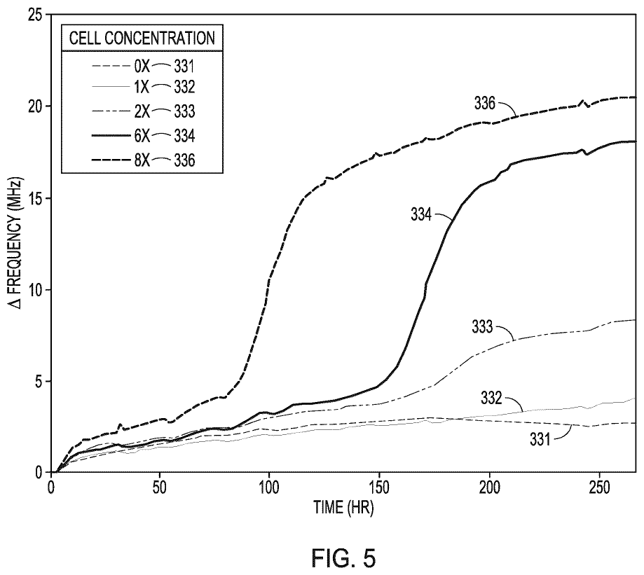

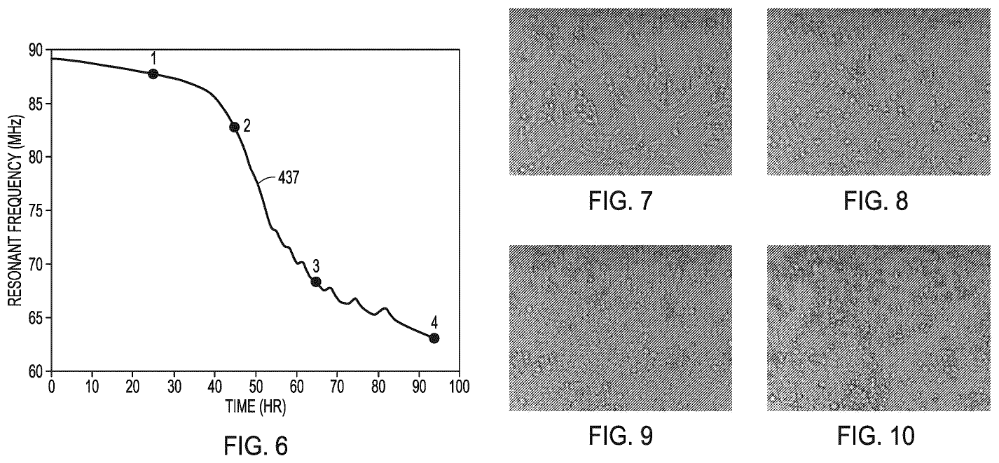

FIG. 5 illustrates changes in resonant frequency over time when sensor systems, having a resonant sensor and a cell-responsive layer to the resonant sensor, were cultured with varying cell seeding concentrations in a container containing the resonant sensor and the cell-responsive layer to the resonant sensor. Multiple sensor system setups were tested with varied cell concentrations. Curve 331 is for a control test with no cells (labelled 0x), which can provide a baseline of the change of resonant frequency over time of the sensor-cell-responsive layer combination. Curve 332 is for a test with a first level of concentration of cells (labelled 1x). Curve 333 is for a test with a second level of concentration of cells (labelled 2x). The second level of concentration of cells is about twice the first level of concentration. Curve 334 is for a test with a third level of concentration of cells (labelled 6x). The third level of concentration of cells is about six times the first level of concentration. Curve 336 is for a test with a fourth level of concentration of cells (labelled 8x). The fourth level of concentration of cells is about eight times the first level of concentration. As the cells were growing for multiple days, the resonant frequency started to vary for the setup with highest cell concentration, corresponding to curve 336. This trend was consistent with the other cell concentrations and the control with no cells (0x) corresponding to curve 331. With this interesting result, a microscope was integrated into the sensor system, as shown in FIG. 4, to visualize the cell growth and correlate to the resonant frequency. From microscope images, the resonant frequency appeared to start changing during the exponential growth and start flattening when the growth slowed down as shown in FIGS. 6-10.

FIGS. 6-10 shows changes in resonant frequency correlated with cell images obtained from a microscope. The images have a field of view of 600×450 μm². FIG. 6 is a plot of resonant frequency as a function of time shown as curve 437. Four times are identified as 1, 2, 3, and 4. FIG. 7 is a cell image at time 1 of FIG. 6. FIG. 8 is a cell image at time 2 of FIG. 6. FIG. 9 is a cell image at time 3 of FIG. 6. FIG. 10 is a cell image at time 4 of FIG. 6.

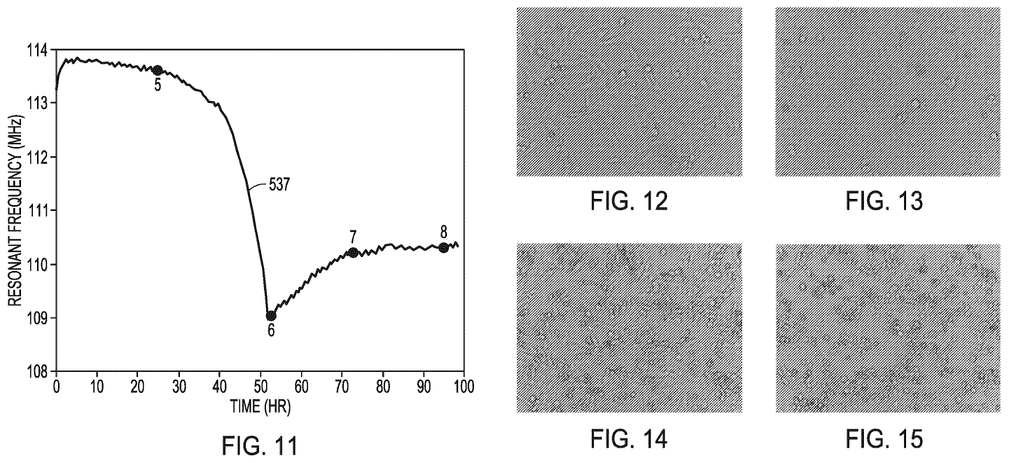

FIGS. 11-15 shows changes in resonant frequency correlated with cell images obtained from a microscope. To investigate the inhibition effect of cell growth on the sensor response, HeLa cells were first grown until a drastic resonant frequency shift was observed. FIG. 11 is a plot of resonant frequency as a function of time shown as curve 537. Four times are identified as 5, 6, 7, and 8. FIG. 12 is a cell image at time 5 of FIG. 11. Niclosamide was introduced at time 6 of FIG. 11 into the culture media at 15 μM. The resonant frequency exhibited almost an instantaneous stop in the resonant frequency shift. Microscopic images have also confirmed the stop of cell growth after introducing the drug. FIG. 13 is a cell image at time 6 of FIG. 11. FIG. 14 is a cell image at time 7 of FIG. 11. FIG. 15 is a cell image at time 8 of FIG. 11.

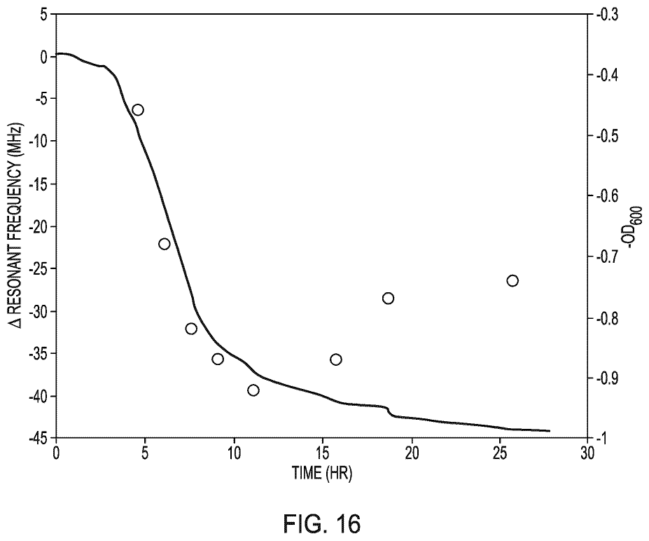

FIG. 16 illustrates changes in resonant frequency over time, benchmarked against a conventional metric. The sensor prototype has shown to be functional to many cell lines including HeLa, HEK293, K562, Jurkat, and CHO cells. In addition to eukaryotes, the sensor also works well with prokaryotes. When the sensor was cultured with E. coli, the resonant frequency also shifted in the similar way to the HeLa growth. When benchmarked against an optical density (OD) conventional metric (OD₆₀₀), the resonant frequency correlates well with the optical density changes.

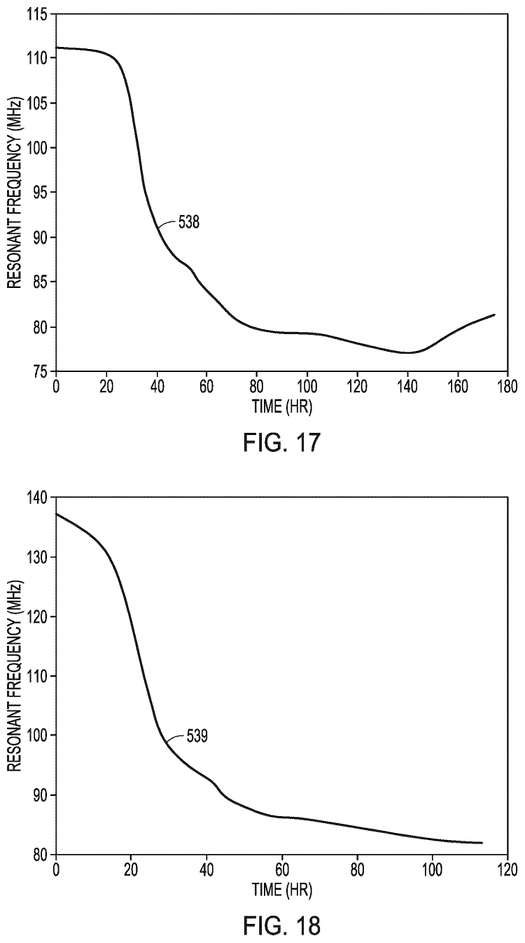

FIGS. 17-18 show results of further investigation performed on cell types and cell-responsive layer thickness. FIG. 17 shows resonant frequency response as a function of time, as curve 538, to the growth of human embryonic kidney (HEK) cell cultures. Similarly, the observation on the resonant frequency changes persisted when tested with HEK cells. FIG. 18 shows resonant frequency response, as curve 539, when a thicker cell-responsive substrate was used. The thicker cell-responsive substrate was an acrylic adhesive transfer tape. The thicker tape results in even more frequency shift. The inventors have hypothesized that the cells secreted molecules that interacts with the cell responsive layer either chemically or physically, which then induced morphological changes of the substrate that results in the change in resonant frequency of the resonant sensor system having a resonant sensor and a cell-responsive layer. It can be seen that this mechanism occurs even without cells growing directly on top of the resonant sensor. Based on pre and post analysis of the resonant sensor having a copper coil, there are morphology changes in which the tape flows into air gap voids associated with the copper coil. This tape flow appears to be a cause of the large change. The cause of this morphological change may likely be some byproduct of cell growth. Further analysis can be conducted to justify the hypothesized mechanism. However, the novel structure is not limited a particular hypothesis

The inventors have discovered that a cell-responsive substrate can enhance the signal change of a resonant sensor, which includes the cell-responsive substrate, in response to secreted molecules by the cells. This structure adds another non-invasive characterization technique for research and manufacturing purposes. Further analysis can be conducted to elucidate the mechanism. The cell-responsive substrate is not limited to the adhesive tapes used in the examples discussed herein. Other potential cell-responsive layers can include, but are not limited to, such as materials as Matrigel™. The cell-responsive substrate can be a cell responsive polymer layer inside the vessel that can be sterilized and does not inhibit cell growth.

A resonant sensor system having a resonant sensor and a cell-responsive layer can be defined by the arrangement of the resonant sensor and the cell-responsive layer with a container in which the resonant sensor and the cell-responsive layer are structured. The arrangement can include voids or air gaps, which can be distributed between or among the resonant sensor and the cell-responsive layer. A void is a volume having boundaries, where within the boundaries of the void there is no solid or liquid material. The void can be a vacuum or filled with a gas. The gas can be from the environment in which the arrangement is made. An air gap is a void filled with air.

The resonant sensor can be implemented in a variety of circuit forms. For example, such a circuit can be an inductor in parallel with a capacitor. The circuit can be a conductive region on a non-shorting surface. Material of the resonant sensor can be selected as one or more conductive materials, such as but not limited to metals. Such metals can include, but is not limited to, copper, silver, gold, cobalt, or iron. The resonant sensor can include an inductive element and a capacitive element. The resonant sensor can include an inductor realized as wire structured as a toroid or laid out flat. The inductor can be a conductive structure arranged as an electrically conducting coil, which can be a copper coil, though other materials may be used to construct the coil. A resonant sensor can be constructed using screen printing to place a conductive paste on a non-shorting substrate, etching a metal such as copper on a polyimide, winding a metal wire into laser-cut acrylic, or other mechanism.

A metal-clad laminate, such as but not limited to a copper-clad laminate, can be used. An example of a copper-clad laminate as a resonant sensor is a thin layer of copper on a layer of polyimide. Such a copper-clad laminate can be a pyralux material. A metal-clad laminate can be implemented without the same height features as a larger copper coil. Voids can be artificially made by cutting into a top layer of the copper-clad laminate, where the top layer can be an acrylic. Dielectric material between loops of the coil can provide capacitance for a resonant sensor. The coil can be an Archimedean coil. The cell-responsive layer can be on top of the inductor. When the inductor is formed flat such that air gaps in the inductor are effectively removed, a signal response to an interrogating signal is not detected. The resonant sensor of the combination of a resonant sensor and cell-responsive layer can be structured with the resonant sensor structured as a coil having a thickness greater than a threshold for producing a resonance signal when interrogated.

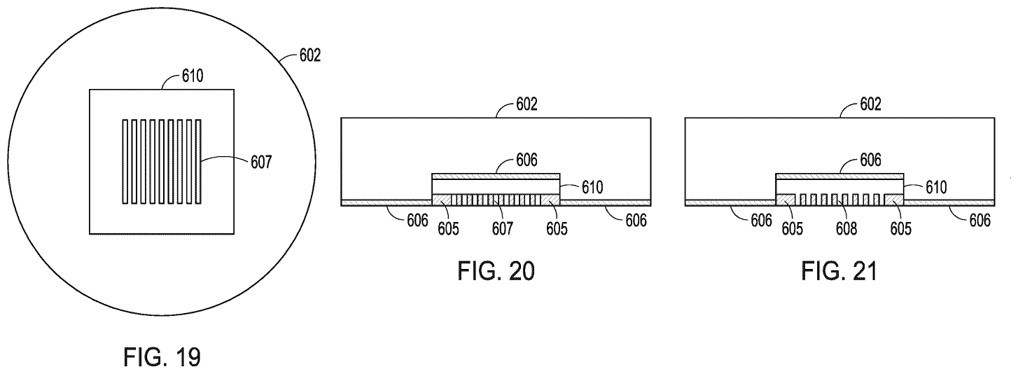

FIG. 19 is a top view of a sensor prototype having a petri dish 602 as a container for a sensor system. This view illustrates a cell-responsive layer 610 over laser cut gaps 607 of a resonant sensor. FIG. 20 is a side view of the sensor prototype in petri dish 602 of FIG. 19 at an initial time when a layer of cells 606 is placed on cell-responsive layer 610 and petri dish 602. Cell-responsive layer 610 is located on a resonant sensor 605, where resonant sensor 605 has laser cut gaps 607. FIG. 21 is the side view of FIG. 20 after a period of cell growth. Cell-responsive layer 610 has conformed into the laser cut gaps 607 during the growth, forming portions 608 of cell-responsive layer 610 in resonant sensor 605. Formation of portions 608 of cell-responsive layer 610 in resonant sensor 605 can provide changes in resonant frequency monitored from interrogation by an interrogator external to the sensor prototype.

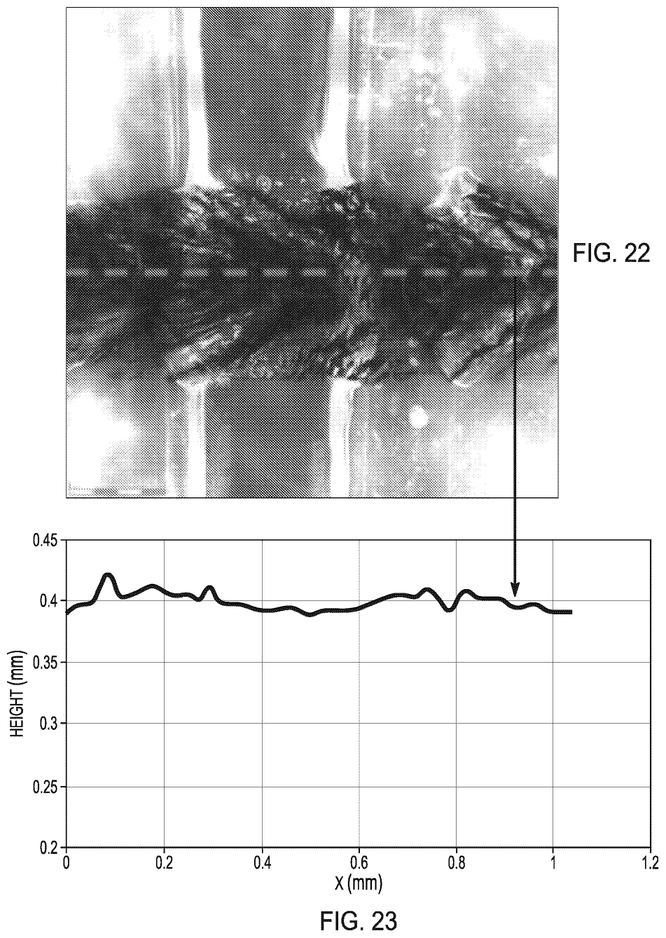

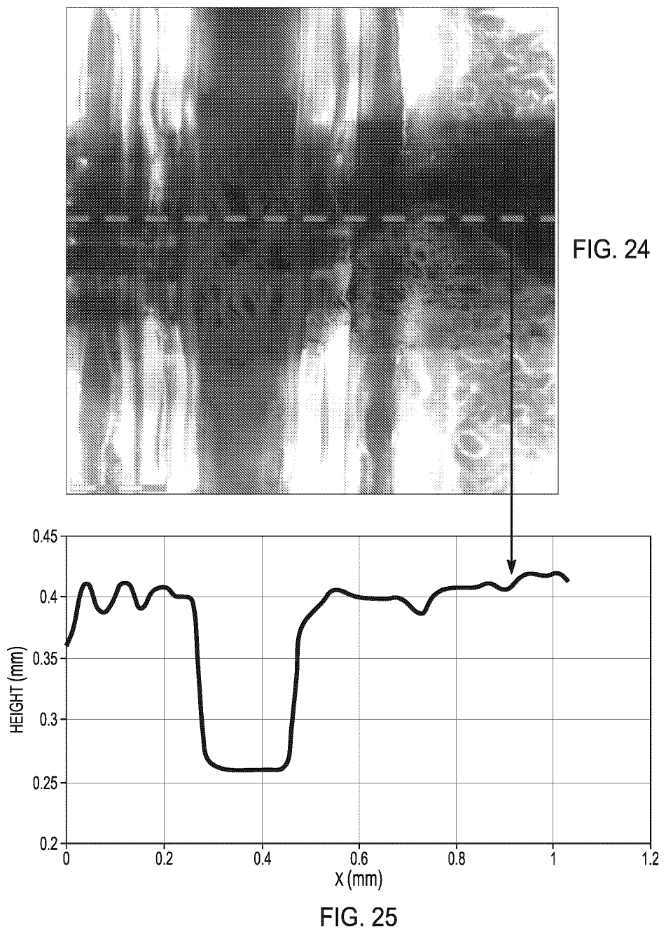

FIG. 22 is a top-down image from a digital microscope of a tape stretched tightly across a gap, without cells, corresponding to a laser cut gap 607 of FIG. 20. FIG. 23 shows height information of tape over laser cut gap 607. FIG. 24 is a top-down image from the digital microscope of the tape upon cell exposure (cell growth), which shows the tape sagging into laser cut gap 607. FIG. 25 shows height information of tape over laser cut gap 607 that reflects the sagging of the tape of FIG. 24.

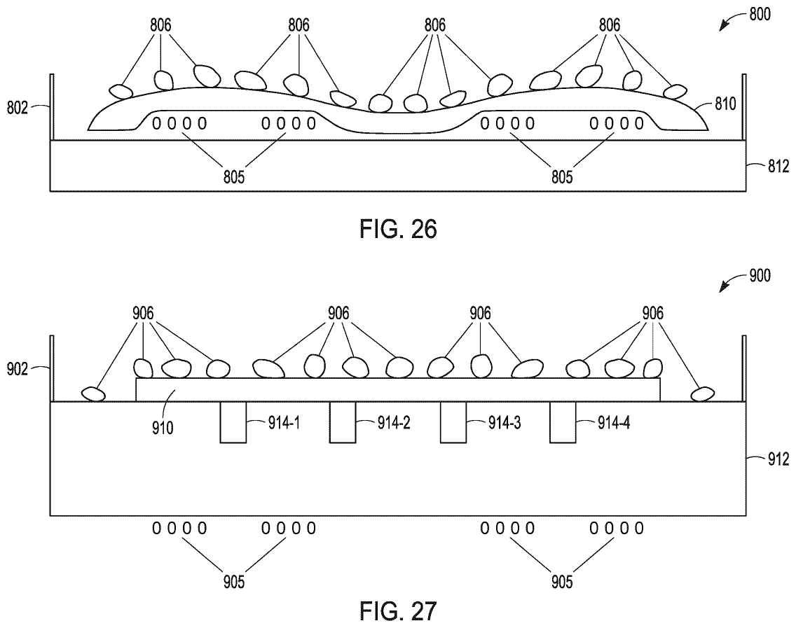

FIG. 26 shows an arrangement 800 of a resonant sensor and a cell-responsive layer to provide enhanced sensitivity of the resonant sensor in cell detection. A sensor coil 805 is formed on a substrate 812 in a container 802. Substrate 812 can be the bottom of container 802 or an impermeable material. A cell-responsive layer 810 is formed over sensor coil 805. Cell-responsive layer 810 can be formed conformally over sensor coil 805. Cell-responsive layer 810 is a material that can interact or allow material of cells to be absorbed when cells 806 are entered in container 802 in proximity to cell-responsive layer 810. Proximity to cell-responsive layer 810 can include contact with cell-responsive layer 810. Cell-responsive layer 810 can be a material that responds to cell growth and can change in its properties. Such changes can include, for example, changes of elasticity that wraps around at least portion of wound copper coil 805 and, therefore, changes the signal provided in response to an interrogation by an external source. The conformal positioning of cell-responsive layer 810 can be structured such that air gaps are maintained in the structure that contribute to a measurement conducted by interrogating arrangement 800. Substrate 812 may contribute to the resonant sensor provided by combination of sensor coil 805 and cell-responsive layer 810, where such contribution by substrate 812 may be part of a baseline measurement. Depending on the nature of the cells being measured, arrangement 800 can be structured without container 802. For example, the cells being measured may be introduced to adhere to and remain on cell-responsive layer 810 without flowing off cell-responsive layer 810.

FIG. 27 shows another arrangement 900 of a resonant sensor and cell-responsive layer to provide enhanced sensitivity of the resonant sensor in cell detection. A sensor coil 905 is located under a container 902 below a substrate 912 in a container 902. Sensor coil 905 can be attached to container 902. Substrate 912 can be the bottom of container 902 or an impermeable material. Substrate 912 can include indentations 914-1, 914-2, 914-3, and 914-4, which are gaps, where the indentations can be air gaps. Though four indentations are shown, substrate 912 can have one or more indentations, where the one or more indentations are more or fewer than four. A cell-responsive layer 910 is formed on substrate 912. Cell-responsive layer 910 can be a material that can interact or allow material of cells to be absorbed when cells 906 are entered in container 902 in proximity to cell-responsive layer 910. Proximity to cell-responsive layer 910 can include contact with cell-responsive layer 910. The interaction of cells with cell-responsive layer 910 can be structured such that contents of one or more indentations 914-1, 914-2, 914-3, and 914-4 are alternated. For container 902 being different from substrate 912, container 902 may contribute to the resonant sensor provided by combination of sensor coil 905, cell-responsive layer 910, and substrate 912, where such contribution by container 902 may be part of a baseline measurement. Depending on the nature of the cells being measured, arrangement 900 can be structured without container 902. For example, the cells being measured may be introduced to adhere to and remain on cell-responsive layer 910 without flowing off cell-responsive layer 910.

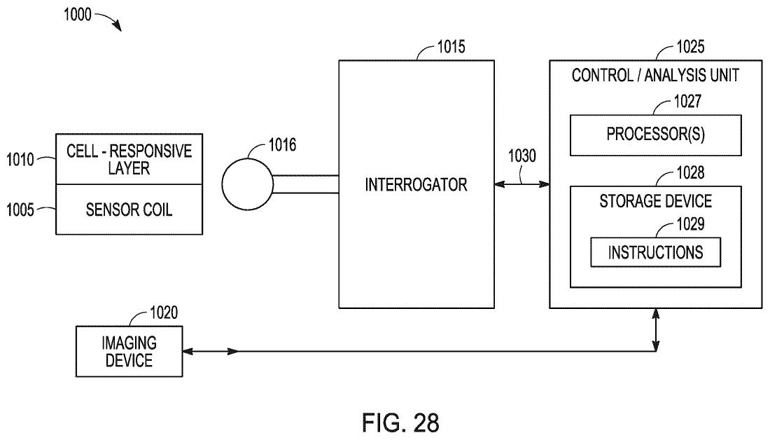

FIG. 28 is a block diagram of an embodiment of an example system architecture 1000 to provide enhanced sensitivity of a resonant sensor structure in cell detection. System architecture 1000 can be operated to wirelessly interrogate cells using a sensor coil 1005 and a cell-responsive layer 1010, where the cells under examination contact cell-responsive layer 1010 or are in proximity of the contact cell-responsive layer 1010 to affect measurement of the resonant frequency of the combination of sensor coil 1005 and cell-responsive layer 1010. The cells may be introduced into a container containing sensor coil 1005 and cell-responsive layer 1010. Depending on the nature of the cells being measured, sensor coil 1005 and cell-responsive layer 1010 can be used without a container over than a platform for sensor coil 1005 and cell-responsive layer 1010, the platform structured depending on a selected structure of sensor coil 1005 and cell-responsive layer 1010 as taught herein. For example, the cells being measured may be introduced to adhere to and remain on cell-responsive layer 1010 without flowing off cell-responsive layer 1010.

Sensor coil 1005 can be structured as a conductive structure shaped to provide an inductor with dielectric, such as air, one or more solid dielectrics, or combinations thereof, between portions providing a capacitor element such that sensor coil 1005 is a resonant sensor. Sensor coil 1005 can be structured in other forms of an antenna structure other than a coil that can provide an inductance and capacitance that can be interrogated using a source external to the arrangement of sensor coil 1005 and cell-responsive layer 1010. Sensor coil 1005 can be a simple circuit that has an inductor in parallel with a capacitor. The inductor can be a looped or zig-zagged conductive trace with the capacitor being either a large, single element placed in parallel or can be composed of many small, capacitive regions that are present in the interstitial spaces of the inductor trace. Sensor coil 1005 and cell-responsive layer 1010 can be structured having a resonant frequency that be measured providing a baseline for measuring cells to be introduced to cell-responsive layer 1010 of arrangement 1000. Sensor coil 1005 and cell-responsive layer 1010 can be structured as taught herein.

Cells introduced to cell-responsive layer 1010 can be monitored over time. The monitoring can be performed by interrogating sensor coil 1005 and cell-responsive layer 1010. Sensor coil 1005 and cell-responsive layer 1010 can be wirelessly interrogated by an interrogator 1015 having an antenna 1016. Antenna 1016 can be a single loop antenna. Other arrangements of antennas, such as multiple antennas, can be used, for example a dual loop antenna set can be used. Wireless interrogation is an electromagnetic probing of an entity without using electrical connections to the entity. A frequency spectrum can be transmitted from antenna 1016 to the combination of sensor coil 1005 and cell-responsive layer 1010 and returned frequencies from sensor coil 1005 and cell-responsive layer 1010 can be received at antenna 1016. The generation of the frequency spectrum and processing of the returned frequencies can be performed by interrogator 1015.

Interrogator 1015 can be a network analyzer. The network analyzer can be a standard vector network analyzer (VNA), which measures signals in terms of scattering parameters. The scattering parameters include parameters for reflected signal, S11, transmitted signal, S21, and reverse parameters, S22 and S12. The resonant frequency of the combination of sensor coil 1005 and cell-responsive layer 1010 can be monitored via interrogator 1015 to transmit a frequency spectrum and to monitor the returned frequencies. This arrangement measures the magnitude and phase of scattered and absorbed frequencies, namely the S11 and S21 scattering parameters. By recording these signals, clear resonant signal features, which are peaks and troughs, are observed and their modulations are observed for sensor readout. Monitored signals from the combination of sensor coil 1005 and cell-responsive layer 1010 can be normalized based on their start frequency and extent of modulation. Alternatively, rather fixed frequency thresholds can be used.

Status of the cells can be correlated to images of the cells at various times after introduction of the cells to cell-responsive layer 1010. System architecture 1000 can include an imaging device 1020 to generate the images of the cells. Imaging device 1020 can be implemented with a microscope or other imaging device. Imaging device 1020 can coupled to a control and analysis unit 1025 of system architecture 1000. Imaging device 1020 can be arranged in various orientations with respect to sensor coil 1005 and cell-responsive layer 1010, depending on the structure of sensor coil 1005 and cell-responsive layer 1010 and the platform on which sensor coil 1005 and cell-responsive layer 1010 is located.

Control and analysis unit 1025, which can include an algorithm for tracking changes in resonant signatures from sensor coil 1005 and cell-responsive layer 1010. Control and analysis unit 1025 can include one or more processors 1027 and a storage device 1028. Storage device 1028 can store instructions 1029 for interrogating the combination of sensor coil 1005 and cell-responsive layer 1010 without cells introduced to cell-responsive layer 1010 and for varying concentrations of cells introduced to cell-responsive layer 1010. Storage device 1028 can store data providing parameters for system architecture 1000 and data from interrogating the combination of sensor coil 1005 and cell-responsive layer 1010. Storage device 1028 can include a digital library of parameters for system architecture 1000 and components of system architecture 1000. Storage device 1028 can be implemented as a group of memory devices to store data electronically. Such memory devices may be arranged as a distributed storage device, which may include remote memory devices accessed over the Internet or other network.

Instructions 1029 of storage device 1028 can include instructions, which when executed by the one or more processors 1025, that cause the system to perform operations to interrogate the resonant sensor system of sensor coil 1005 and cell-responsive layer 1010 with varying concentrations of cells at a number of different times using antenna 1016 or one or more antennas and a network analyzer implemented in interrogator 1015. The operations can include operations to monitor the resonant frequency of the combination of sensor coil 1005 and cell-responsive layer 1010 from the interrogation at each time of the number of different times. The operations can include operations to evaluate status of the cells from the monitored resonant frequencies. Operations to evaluate the status of the cells can include operations to identify changes in the monitored resonant frequency as a function of time and to correlate identified changes to the cells. Identified changes to the cells can be correlated using imaging device 1020.

The operations can include operations to scan the sensor system of sensor coil 1005 and cell-responsive layer 1010 combination to measure changes of resonant frequency as a function of time correlated to the concentration of cells introduced to cell-responsive layer 1010. The scan can use antenna 1016 and interrogator 1015 realized by a network analyzer to detect a phase and a magnitude of each of a S11 scattering parameter (reflection) and a S21 scattering parameter (transmission), which can provide four vectors for analysis. Both the S11 and S21 scattering parameters can be detected from sensor coil 1005 and cell-responsive layer 1010 using a VNA, where S22 and S12 scattering parameters are neglected as they are symmetric to S11 and S21, respectively. Multiple signal features, such as peak frequency, width, and height can be used to perform principal component analysis (PCA) and deconvolute the data. Multivariate regression of the four vectors may also be used in analysis of the response.

Control and analysis unit 1025 can be implemented to automate scanning of all scattering parameters (S11, S22, S12, and S21 magnitude and phase). Antennas can be coupled to a laptop for data acquisition and control. Interrogator 1015, such as a VNA, can be operated without a monitor or graphical user interface (GUI) by controlling interrogator 1015 with appropriate electronics. Control and analysis unit 1025 can analyze signal changes in polar coordinates to perform signal analysis utilizing both magnitude and phase of the scattering parameter. Other coordinate systems can be used. An algorithm in control and analysis unit 1025 can track the modulation extent of the scattering parameter signals and normalize the response based on start signals, as each arrangement of sensor coil 1005 and cell-responsive layer 1010 can have a different start frequency due to different structures of sensor coil 1005 and cell-responsive layer 1010 and variations in fabrication. This normalized modulation extent can be used to correlate to the concentration of cells.

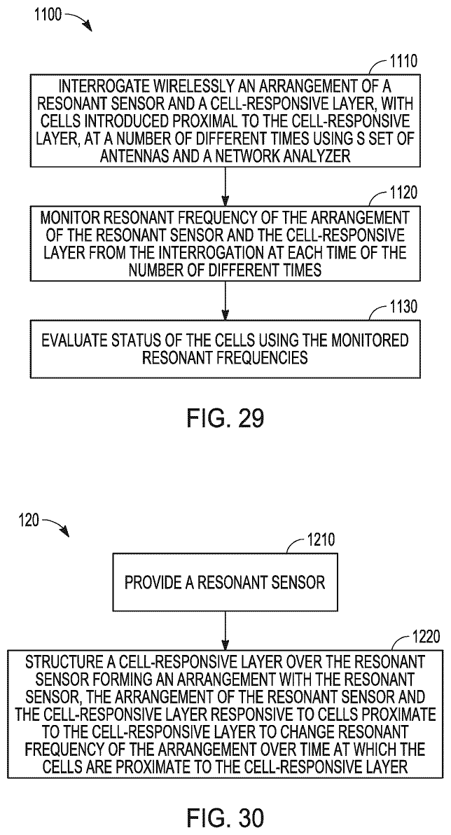

FIG. 29 is a flow diagram of features of an example embodiment of a method 1100 of measuring cells. At 1110, an arrangement of a resonant sensor and a cell-responsive layer, with cells introduced proximal to the cell-responsive layer, is interrogated at a number of different times using a set of antennas and a network analyzer. At 1120, resonant frequency of the arrangement of the resonant sensor and the cell-responsive layer is monitored from the interrogation at each time of the number of different times. At 1130, status of the cells is evaluated using the monitored resonant frequencies. Alternatively or in conjunction with evaluating monitored resonant frequencies, peak magnitude or power reflection/transmission levels can be evaluated.

Variations of method 1100 or methods similar to method 1100 can include a number of different embodiments that may be combined depending on the application of such methods and/or the architecture of systems in which such methods are implemented. Such methods can include identifying changes in the monitored resonant frequency as a function of time and correlating the identified changes to images of the cells to evaluate the status.

Variations of method 1100 or methods similar to method 1100 can include the arrangement of a resonant sensor and a cell-responsive layer being structured in various formats. The arrangement of a resonant sensor and a cell-responsive layer can be structured inside a vessel with the resonant sensor being a conductive coil attached to an inner bottom of the vessel by the cell-responsive layer. The cell-responsive layer can be structured conformally on the resonant sensor, with voids distributed in the arrangement of the resonant sensor and the cell-responsive layer. The cell-responsive layer can be structured with a substrate positioned between the resonant sensor and the cell-responsive layer. The substrate can have gaps in a first surface of the substrate, where the first surface is opposite a second surface of the substrate. The resonant sensor can be positioned under the second surface. Variations can include the cell-responsive layer being positioned on and contacting the first surface of the substrate.

In various embodiments, a machine-readable storage device can comprise instructions, which, when executed by one or more processors, cause a machine to perform operations to perform functions associated with any features associated with method 1100, variations of method 1100, or methods similar to method 1100.

In various embodiments, an apparatus can comprise a resonant sensor and a cell-responsive layer structured over the resonant sensor in an arrangement with the resonant sensor. The arrangement of the resonant sensor and the cell-responsive layer is responsive to cells proximate to the cell-responsive layer to change resonant frequency of the arrangement over time at which the cells are proximate to the cell-responsive layer. Cells proximate to the cell-responsive layer can be cells contacting the cell-responsive layer.

Variations of such an apparatus or similar apparatus can include a number of different embodiments that may be combined depending on the application of such apparatus and/or the architecture of systems in which such apparatus are implemented. Such apparatus can include features of the arrangement of the resonant sensor and the cell-responsive layer being structured in various formats. The cell-responsive layer can be a polymer layer that does not inhibit cell growth. The cell-responsive layer can be a sterilizable material that maintains responsiveness to cells after sterilization. The cell-responsive layer can have an adhesive property. The resonant sensor and the cell-responsive layer can be structured inside a vessel with resonant sensor being a conductive coil attached to an inner bottom of the vessel by the cell-responsive layer. The cell-responsive layer can be structured conformally on the resonant sensor, with voids distributed in the arrangement of the resonant sensor and the cell-responsive layer such that upon cell growth portions of the cell-responsive layer conforms to the resonant sensor or fills the voids. The voids can be air gaps. A substrate can be positioned between the resonant sensor and the cell-responsive layer. The substrate can have gaps in a first surface of the substrate, where the first surface is opposite a second surface of the substrate, the resonant sensor being positioned under the second surface. The cell-responsive layer can be positioned on and contacting the first surface of the substrate. The resonant sensor can include a copper coil.

In various embodiments, a machine-readable storage device can comprise instructions, which, when executed by one or more processors, cause a machine to perform operations to perform functions associated with any features associated with such apparatus, variations of such apparatus, or apparatus similar to such apparatus.

In various embodiments, a system can comprise a resonant sensor, a cell-responsive layer structured over the resonant sensor in an arrangement with the resonant sensor, a set of antennas, and a network analyzer. The arrangement of the resonant sensor and the cell-responsive layer is responsive to cells proximate to the cell-responsive layer to change resonant frequency of the arrangement over time at which the cells are proximate to the cell-responsive layer. The set of antennas are arranged to wirelessly interrogate the arrangement of the resonant sensor and detect signals from the arrangement of the resonant sensor and the cell-responsive layer in response to wireless interrogation. The set of antennas is a set of one or more antennas. The network analyzer is coupled to the antenna to control interrogation of the arrangement of the resonant sensor and the cell-responsive layer and analyze detected signals from the set of antennas.

Variations of such a system or similar system can include a number of different embodiments that may be combined depending on the application of such systems and/or the architecture in which such systems are implemented. Such systems can include a number of features. The system can include an imaging device positioned to image the cells proximate to the cell-responsive layer. The imaging device include, but is not limited to, a microscope. The network analyzer can be a vector network analyzer.

Variations of such a system or similar system can include features of the arrangement of the resonant sensor and the cell-responsive layer being structured in various formats. The cell-responsive layer can be a polymer layer that does not inhibit cell growth. The cell-responsive layer can be a sterilizable material that maintains responsiveness to cells after sterilization. The cell-responsive layer can have an adhesive property. The resonant sensor and the cell-responsive layer can be structured inside a vessel with the resonant sensor being a conductive coil attached to an inner bottom of the vessel by the cell-responsive layer. The cell-responsive layer can be structured conformally on the resonant sensor, with voids distributed in the arrangement of the resonant sensor and the cell-responsive layer. The voids can be air gaps. A substrate can be positioned between the resonant sensor and the cell-responsive layer. The substrate can have gaps in a first surface of the substrate, where the first surface is opposite a second surface of the substrate. The resonant sensor can be positioned under the second surface. The cell-responsive layer can be positioned on and contacting the first surface of the substrate. The resonant sensor can include, but is not limited to, a copper coil.

Variations of such a system or similar system can include one or more processors and a storage device comprising instructions, which when executed by the one or more processors, cause the system to perform operations. The operations can include operations to interrogate the arrangement of the resonant sensor and the cell-responsive layer, with cells introduced proximal to the cell-responsive layer, at a number of different times using the set of antennas. The operations can include operations to monitor resonant frequency of the arrangement of the resonant sensor and the cell-responsive layer from the interrogation at each time of the number of different times. The operations can include operations to evaluate status of the cells from the monitored resonant frequencies. The operations to evaluate the status of the cells can include operations to identify changes in the monitored resonant frequency as a function of time and to correlate the identified changes to images of the cells obtained from an imaging device of the system.

In various embodiments, a machine-readable storage device can comprise instructions, which, when executed by one or more processors, cause a machine to perform operations to perform functions associated with any features associated with such systems, variations of such systems, or systems similar to such apparatus.

FIG. 30 is a flow diagram of features of an example embodiment of a method 1200 of structuring a resonant sensor and a cell-responsive layer sensor arrangement. At 1210, a resonant sensor having an inductive element and a capacitive element is provided. At 1220, a cell-responsive layer is structured over the resonant sensor forming an arrangement with the resonant sensor. The arrangement of the resonant sensor and the cell-responsive layer is responsive to cells proximate to the cell-responsive layer to change resonant frequency of the arrangement over time at which the cells are proximate to the cell-responsive layer.

Variations of method 1200 or methods similar to method 1200 can include a number of different embodiments that may be combined depending on the application of such methods and/or the architecture of systems for which such methods are implemented. Such methods can include the arrangement of a resonant sensor and a cell-responsive layer being structured in various formats. Structuring the cell-responsive layer can include selecting a polymer layer that does not inhibit cell growth. Structuring the cell-responsive layer can include selecting sterilizable material that maintains responsiveness to cells after sterilization. Structuring the cell-responsive layer can include selecting cell-responsive material having an adhesive property. Structuring the cell-responsive layer over the resonant sensor can include attaching a conductive coil to an inner bottom of an vessel by the cell-responsive layer. Structuring the cell-responsive layer over the resonant sensor can include conformally structuring the cell-responsive layer on the resonant sensor, with voids distributed in the arrangement of the resonant sensor and the cell-responsive layer. Variations of method 1200 or methods similar to method 1200 can include positioning a substrate between the resonant sensor and the cell-responsive layer, with the substrate having gaps in a first surface of the substrate, the first surface being opposite a second surface of the substrate and positioning the resonant sensor under the second surface. Variations can include positioning the cell-responsive layer on and contacting the first surface of the substrate.

The following are example apparatus, systems, and methods, in accordance with the teachings herein.

An example apparatus 1 can comprise: a resonant sensor; and a cell-responsive layer structured over the resonant sensor in an arrangement with the resonant sensor, the arrangement of the resonant sensor and the cell-responsive layer responsive to cells proximate to the cell-responsive layer to change resonant frequency of the arrangement over time at which the cells are proximate to the cell-responsive layer.

An example apparatus 2 can include features of example apparatus 1 and can include the cell-responsive layer being a polymer layer that does not inhibit cell growth.

An example apparatus 3 can include features of any features of the preceding example apparatus and can include the cell-responsive layer having a sterilizable material that maintains responsiveness to cells after sterilization.

An example apparatus 4 can include features of any of the preceding example apparatus and can include the cell-responsive layer having an adhesive property.

An example apparatus 5 can include features of any of the preceding example apparatus and can include the resonant sensor and the cell-responsive layer being structured inside a vessel with the resonant sensor being a conductive coil attached to an inner bottom of the vessel by the cell-responsive layer.

An example apparatus 6 can include features of any of the preceding example apparatus and can include the cell-responsive layer being structured conformally on the resonant sensor, with voids distributed in the arrangement of the resonant sensor and the cell-responsive layer.

An example apparatus 7 can include features of example apparatus 6 and any of the preceding example apparatus and can include the voids being air gaps.

An example apparatus 8 can include features of any of the preceding example apparatus 1-4 and can include a substrate being positioned between the resonant sensor and the cell-responsive layer, the substrate having gaps in a first surface of the substrate, the first surface being opposite a second surface of the substrate, the resonant sensor being positioned under the second surface.

An example apparatus 9 can include features of example apparatus 8 and any of the preceding example apparatus 1-4 and can include the cell-responsive layer being positioned on and contacting the first surface of the substrate.

An example apparatus 10 can include features of any of the preceding example apparatus and can include the resonant sensor includes a copper coil.

In an example apparatus 11, any of the apparatus of example apparatus 1 to 10 may include apparatus incorporated into an electronic apparatus further comprising a host processor and a communication bus extending between the host processor and the apparatus.

In an example apparatus 12, any of the apparatus of example apparatus 1 to 11 may be modified to include any structure presented in another of example apparatus 1 to 11.

In an example apparatus 13, any apparatus associated with the apparatus of example apparatus 1 to 12 may further include a machine-readable storage device configured to store instructions as a physical state, wherein the instructions may be used to perform one or more operations of the apparatus.

In an example apparatus 14, any of the apparatus of example apparatus 1 to 13 may be operated in accordance with any of the below example methods 1 to 10 and example methods 11 to 22.

An example system 1 can comprise: a resonant sensor; a cell-responsive layer structured over the resonant sensor in an arrangement with the resonant sensor, the arrangement of the resonant sensor and the cell-responsive layer responsive to cells proximate to the cell-responsive layer to change resonant frequency of the arrangement over time at which the cells are proximate to the cell-responsive layer; a set of antennas arranged to wirelessly interrogate the arrangement of the resonant sensor and detect signals from the arrangement of the resonant sensor and the cell-responsive layer in response to wireless interrogation; and a network analyzer coupled to the antenna to control interrogation of the arrangement of the resonant sensor and the cell-responsive layer and analyze detected signals from the set of antennas.

An example system 2 can include features of preceding example system 1 and can include the resonant sensor to include a metal-clad laminate.

An example system 3 can include features of example system 2 and any of the preceding example systems and can include the metal-clad laminate being a copper-clad laminate.

An example system 4 can include features of any of the preceding example systems and can include the network analyzer being a vector network analyzer.

An example system 5 can include features of any features of the preceding example systems and can include the cell-responsive layer being a polymer layer that does not inhibit cell growth.

An example system 6 can include features of any features of the preceding example systems and can include the cell-responsive layer being a sterilizable material that maintains responsiveness to cells after sterilization.

An example system 7 can include features of any features of the preceding example systems and can include the cell-responsive layer having an adhesive property.

An example system 8 can include features of any features of the preceding example systems and can include the resonant sensor and the cell-responsive layer being structured inside a vessel with the resonant sensor being a conductive coil attached to an inner bottom of the vessel by the cell-responsive layer.

An example system 9 can include features of any features of the preceding example systems and can include the cell-responsive layer being structured conformally on the resonant sensor, with voids distributed in the arrangement of the resonant sensor and the cell-responsive layer.

An example system 10 can include features of example system 9 and any features of the preceding example systems and can include the voids are air gaps.

An example system 11 can include features of any features of the preceding example systems 1 to 8 and can include a substrate being positioned between the resonant sensor and the cell-responsive layer, the substrate having gaps in a first surface of the substrate, the first surface being opposite a second surface of the substrate, the resonant sensor being positioned under the second surface.

An example system 12 can include features of example system 11 and any features of the preceding example systems and can include the cell-responsive layer being positioned on and contacting the first surface of the substrate.

An example system 13 can include features of any features of the preceding example systems and can include the resonant sensor having a copper coil.

An example system 14 can include features of any features of the preceding example systems and can include one or more processors; and a storage device comprising instructions, which when executed by the one or more processors, cause the system to perform operations to: interrogate the arrangement of the resonant sensor and the cell-responsive layer, with cells introduced proximal to the cell-responsive layer, at a number of different times using the set of antennas; monitor resonant frequency of the arrangement of the resonant sensor and the cell-responsive layer from the interrogation at each time of the number of different times; and evaluate status of the cells from the monitored resonant frequencies.

An example system 15 can include features of example system 14 and any features of the preceding example systems and can include the operations to evaluate the status of the cells to include operations to identify changes in the monitored resonant frequency as a function of time and to correlate the identified changes to images of the cells obtained from an imaging device of the system.

In an example system 16, any of the systems of example systems 1 to 15 may include one or more systems further comprising a host processor and a communication bus extending between the host processor and the one or more systems.

In an example system 17, any of the systems of example systems 1 to 16 may be modified to include any structure presented in another of example system 1 to 16.

In an example system 18, any apparatus associated with the systems of example systems 1 to 17 may further include a machine-readable storage device configured to store instructions as a physical state, wherein the instructions may be used to perform one or more operations of the system.

In an example system 19, any of the systems of example systems 1 to 18 may be formed in accordance with any of the methods of the below example methods 1 to 10 and example methods 11 to 22.

An example method 1 can comprise: interrogating wirelessly an arrangement of a resonant sensor and a cell-responsive layer, with cells introduced proximal to the cell-responsive layer, at a number of different times using a set of antennas and a network analyzer; monitoring resonant frequency of the arrangement of the resonant sensor and the cell-responsive layer from the interrogation at each time of the number of different times; and evaluating status of the cells using the monitored resonant frequencies.

An example method 2 can include features of example method 1 and can include evaluating the status to include identifying changes in the monitored resonant frequency as a function of time and correlating the identified changes to images of the cells.

An example method 3 can include features of any of the preceding example methods and can include the arrangement of the resonant sensor and the cell-responsive layer being structured inside a vessel with the resonant sensor being a conductive coil attached to an inner bottom of the vessel by the cell-responsive layer.

An example method 4 can include features of any of the preceding example methods and can include the cell-responsive layer being structured conformally on the resonant sensor, with voids distributed in the arrangement of the resonant sensor and the cell-responsive layer.

An example method 5 can include features of any of the preceding example methods and can include a substrate being positioned between the resonant sensor and the cell-responsive layer, the substrate having gaps in a first surface of the substrate, the first surface being opposite a second surface of the substrate, the resonant sensor being positioned under the second surface.

An example method 6 can include features of example method 5 and any of the preceding example methods and can include the cell-responsive layer being positioned on and contacting the first surface of the substrate.

In an example method 7, any of the example methods 1 to 6 may be performed in operating an electronic apparatus further comprising a host processor and a communication bus extending between the host processor and the memory device.

In an example method 8, any of the example methods 1 to 7 may be modified to include operations set forth in any other of example methods 1 to 7.

In an example method 9, any of the example methods 1 to 8 may be implemented at least in part through use of instructions stored as a physical state in one or more machine-readable storage devices.

An example method 10 can include features of any of the preceding example methods 1 to 9 and can include performing functions associated with any features of example apparatus 1 to 14 and example systems 1 to 19.

An example method 11 can comprise: providing a resonant sensor; and structuring a cell-responsive layer over the resonant sensor forming an arrangement with the resonant sensor, the arrangement of the resonant sensor and the cell-responsive layer responsive to cells proximate to the cell-responsive layer to change resonant frequency of the arrangement over time at which the cells are proximate to the cell-responsive layer.

An example method 12 can include features of example method 11 and can include structuring the cell-responsive layer includes selecting a polymer layer that does not inhibit cell growth.

An example method 13 can include features of any of the preceding example methods and can include structuring the cell-responsive layer to include selecting sterilizable material that maintains responsiveness to cells after sterilization.

An example method 14 can include features of any of the preceding example methods and can include structuring the cell-responsive layer to include selecting cell-responsive material having an adhesive property.

An example method 15 can include features of any of the preceding example methods and can include structuring the cell-responsive layer over the resonant sensor to include attaching a conductive coil to an inner bottom of an vessel by the cell-responsive layer.

An example method 16 can include features of example method 5 and any of the preceding example methods and can include structuring the cell-responsive layer over the resonant sensor to include conformally structuring the cell-responsive layer on the resonant sensor, with voids distributed in the arrangement of the resonant sensor and the cell-responsive layer.

An example method 17 can include features of example methods any of the preceding example methods and can include positioning a substrate between the resonant sensor and the cell-responsive layer, with the substrate having gaps in a first surface of the substrate, the first surface being opposite a second surface of the substrate and positioning the resonant sensor under the second surface.

An example method 18 can include features of example method 17 and any of the preceding example methods 11 to 16 and can include positioning the cell-responsive layer on and contacting the first surface of the substrate.

In an example method 19, any of the example methods 11 to 18 may be performed in forming an electronic apparatus further comprising a host processor and a communication bus extending between the host processor and the memory device.

In an example method 20, any of the example methods 11 to 19 may be modified to include operations set forth in any other of example methods 11 to 19.

In an example method 21, any of the example methods 11 to 20 may be implemented at least in part through use of instructions stored as a physical state in one or more machine-readable storage devices.

An example method 22 can include features of any of the preceding example methods 11 to 21 and can include performing functions associated with any features of example apparatus 1 to 14 and example systems 1 to 19.

An example machine-readable storage device 1 storing instructions, that when executed by one or more processors, cause a machine to perform operations, can comprise instructions to perform functions associated with any features of example apparatus 1 to 14 and example systems 1 to 19 or perform methods associated with any features of example methods 1 to 10 and any features of example methods 11 to 22 .

Although specific embodiments have been illustrated and described herein, it will be appreciated by those of ordinary skill in the art that any arrangement that is calculated to achieve the same purpose may be substituted for the specific embodiments shown. Various embodiments use permutations and/or combinations of embodiments described herein. It is to be understood that the above description is intended to be illustrative, and not restrictive, and that the phraseology or terminology employed herein is for the purpose of description.

1-40. (canceled)

41. An apparatus comprising:
a resonant sensor; and
a cell-responsive layer structured over the resonant sensor in an arrangement with the resonant sensor, the arrangement of the resonant sensor and the cell-responsive layer responsive to cells proximate to the cell-responsive layer to change resonant frequency of the arrangement over time at which the cells are proximate to the cell-responsive layer.

42. The apparatus of claim 41, wherein the cell-responsive layer is a polymer layer that does not inhibit cell growth.

43. The apparatus of claim 41, wherein the cell-responsive layer is a sterilizable material that maintains responsiveness to cells after sterilization.

44. The apparatus of claim 41, wherein the cell-responsive layer is structured conformally on the resonant sensor, with voids distributed in the arrangement of the resonant sensor and the cell-responsive layer.

45. The apparatus of claim 44, wherein voids are air gaps.

46. The apparatus of claim 41, wherein a substrate is positioned between the resonant sensor and the cell-responsive layer, the substrate having gaps in a first surface of the substrate, the first surface being opposite a second surface of the substrate, the resonant sensor being positioned under the second surface.

47. The apparatus of claim 46, wherein the cell-responsive layer is positioned on and contacting the first surface of the substrate.

48. A system comprising:
a resonant sensor;
a cell-responsive layer structured over the resonant sensor in an arrangement with the resonant sensor, the arrangement of the resonant sensor and the cell-responsive layer responsive to cells proximate to the cell-responsive layer to change resonant frequency of the arrangement over time at which the cells are proximate to the cell-responsive layer;
a set of antennas arranged to wirelessly interrogate the arrangement of the resonant sensor and to detect signals from the arrangement of the resonant sensor and the cell-responsive layer in response to wireless interrogation, and
a network analyzer coupled to the antenna to control interrogation of the arrangement of the resonant sensor and the cell-responsive layer and analyze detected signals from the set of antennas.

49. The system of claim 48, wherein the cell-responsive layer is a polymer layer that does not inhibit cell growth and the polymer layer is a sterilizable material that maintains responsiveness to cells after sterilization.

50. The system of claim 48, wherein the resonant sensor and the cell-responsive layer are structured inside a vessel with the resonant sensor being a conductive coil attached to an inner bottom of the vessel by the cell-responsive layer.

51. The system of claim 48, wherein the cell-responsive layer is structured conformally on the resonant sensor, with voids distributed in the arrangement of the resonant sensor and the cell-responsive layer.

52. The system of claim 48, wherein a substrate is positioned between the resonant sensor and the cell-responsive layer, the substrate having gaps in a first surface of the substrate, the first surface being opposite a second surface of the substrate, the resonant sensor being positioned under the second surface.

53. The system of claim 52, wherein the cell-responsive layer is positioned on and contacting the first surface of the substrate.

54. The system of claim 48, wherein the system includes:
one or more processors; and
a storage device comprising instructions, which when executed by the one or more processors, cause the system to perform operations to:
interrogate the arrangement of the resonant sensor and the cell-responsive layer, with cells introduced proximal to the cell-responsive layer, at a number of different times using the set of antennas;
monitor resonant frequency of the arrangement of the resonant sensor and the cell-responsive layer from the interrogation at each time of the number of different times; and
evaluate status of the cells from the monitored resonant frequencies.

55. The system of claim 54, wherein the operations to evaluate the status of the cells include operations to identify changes in the monitored resonant frequency as a function of time and to correlate the identified changes to images of the cells obtained from an imaging device of the system.

56. A method comprising:
interrogating wirelessly an arrangement of a resonant sensor and a cell-responsive layer, with cells introduced proximal to the cell-responsive layer, at a number of different times using a set of antennas and a network analyzer;
monitoring resonant frequency of the arrangement of the resonant sensor and the cell-responsive layer from the interrogation at each time of the number of different times; and
evaluating status of the cells using the monitored resonant frequencies.

57. The method of claim 56, wherein evaluating the status includes identifying changes in the monitored resonant frequency as a function of time and correlating the identified changes to images of the cells.

58. The method of claim 56, wherein the cell-responsive layer is structured conformally on the resonant sensor, with voids distributed in the arrangement of the resonant sensor and the cell-responsive layer.

59. The method of claim 56, wherein a substrate is positioned between the resonant sensor and the cell-responsive layer, the substrate having gaps in a first surface of the substrate, the first surface being opposite a second surface of the substrate, the resonant sensor being positioned under the second surface.

60. The method of claim 59, wherein the cell-responsive layer is positioned on and contacting the first surface of the substrate.
